(19) United States
(12) Patent Application Publication
(10) Pub. No.: US 2021/0338546 A1
(43) Pub. Date: Nov. 4, 2021

(54) HAND CLEANING COMPOSITION WITH TIMED USE DURATION

(71) Applicant: WeCool Toys, Inc., Point Pleasant, NJ (US)

(72) Inventors: Jeffrey Osnato, Masnasquan, NJ (US); Katherine Rae Carey, Manasquan, NJ (US)

(21) Appl. No.: 17/306,293

(22) Filed: May 3, 2021

Related U.S. Application Data
(60) Provisional application No. 63/019,519, filed on May 4, 2020.

(51) Int. Cl. A61K 8/18; A61Q 17/00; A61Q 19/10

(52) U.S. Cl. CPC A61K 8/18; A61Q 17/005; A61K 2800/80; A61K 2800/74; A61Q 19/10

(57) ABSTRACT

There is provided a composition and method of use in cleansing skin, wherein the volume of the composition is provided in an amount correlated to a desired wash duration, preferably of 20 seconds or greater. Further, the composition and method of us thereof provide the composition is exhausted within a desired wash duration, beginning when the composition is first contacted with water.

### CROSS-REFERENCE TO RELATED APPLICATIONS

This application claims priority to U.S. Provisional Patent Application No. 63/019,519 which was filed on May 4, 2020.

### BACKGROUND OF THE DISCLOSURE

The disclosure to which this application relates is to a composition and a method of use therof of a skin cleaning and/or sanitizing composition with a timed duration of use.

In particular, although not necessarily exclusively, the composition and method to which the application relates is a soap composition for use in hand-washing and/or cleaning, and is herein referred to in a non-limiting manner as a soap composition. The soap compositions of this type conventially include sanitizing and cleaning compounds suitable for the killing of bacteria, viruses, and the like, as well as the removal of dirt or other impurities, while still providing adjuvant properties, such as skin care (lotion, hydration, etc.).

While it is well known that such soap compositions are traditionally used for hand washing and cleaning, particular contexts require a duration of washing in order to effectively kill germs, bacteria, viruses, and the like and provide the requiste level of sanitization. However, despite the cleaning advantages of the soap, there exists problems with properly dispensing the required amount of soap or sanitizing composition to achieve the desired level of cleanliness and/or sanitization. Therefore, it is the aim of the disclosure to provide a composition and a method of use that provides a desired amount of soap composition for the required duration of cleaning.

### DETAILED DESCRIPTION

Although this disclosure has been described relative to specific materials, it is not limited to the specific materials or apparatuses but only to their specific characteristics and capabilities and other materials and apparatuses can be substituted as is well understood by those skilled in the art after appreciating the present disclosure.

It is to be understood that the terms such as left, right, top, bottom, front, back, side, height, length, width, upper, lower, interior, exterior, inner, outer, and the like as may be used herein, merely describe points of reference and do not limit the present disclosure to any particular orientation or configuration.

As used herein, the singular forms “a,” “an,” and “the” are intended to include both the singular and plural forms, unless the language explicitly indicates otherwise. Indefinite articles like “a” and “an” introduce or refer to any modified term, both previously-introduced and not, while definite articles like “the” refer to a same previously-introduced term; as such, it is understood that “a” or “an” modify items that are permitted to be previously-introduced or new, while definite articles modify an item that is the same as immediately previously presented. It will be further understood that the terms “comprises,” “comprising,” “includes,” and/or “including,” when used herein, specify the presence of stated features, characteristics, steps, operations, elements, and/or components, but do not themselves preclude the presence or addition of one or more other features, characteristics, steps, operations, elements, components, and/or groups thereof.

### A Santizing Composition with Timed Duration of Use

According to an embodiment of the disclosure, any suitable soap, santizer, or antimicrobial composition may be formulated for use as a skin cleaning or washing agent.

In a preferred embodiment of the disclosure, the soap composition is a solid, though a liquid, foam, gel, powder, or other fomulation may be used as well. In a still further embodiment of the disclosure, the soap composition is provided in a volume that correlates to the duration of washing/cleaning time required by the cleaning context.

In an embodiment, the volume of soap composition provided is suitable for a washing duration of 10 seconds, 20 seconds, 30 seconds, 60 seconds, 2 minutes, 5 minutes, 7 minutes, or 10 minutes, preferably greater than 20 seconds. However, the volume of soap composition may be modified to be suitable for any duration without departing from the disclosure. For purposes of clarity, washing duration is defined as beginning from the first contact with skin and/or water.

In an aspect of the disclosure, the soap composition is completely exhausted, dissolved, or expended during the desired duration of washing, thus signaling the end of the washing period, wherein the soap composition may then be rinsed from the skin.

It should be appreciated that the formulation or composition can take any suitable form and may be comprised of various componets, as required to achieve the desired results.

There it is therefore provided in accordance with the disclosure, a soap composition which is formulated and dispensed at a volume to achieve the desired duration of cleaning, washing, and/or santitizing.

What is claimed:

1. A composition for use in cleansing skin comprising a solid volume of soap, wherein the volume of the composition is exhausted within a particular wash duration.

2. The composition of claim 1 wherein the composition further comprises an antimicrobial or sanitizing composition.

3. The composition of claim 1 wherein the volume of the composition is exhausted within a 20 second wash duration, wherein wash duration begins when the composition is contacted with water.

4. The composition of claim 1 wherein the volume of the composition is exhausted within a 60 second wash duration, wherein wash duration begins when the composition is contacted with water.

5. The composition of claim 1 wherein the volume of the composition is exhausted within a 5 minute wash duration, wherein wash duration begins when the composition is contacted with water.

6. The composition of claim 1 wherein the volume of the composition is exhausted within a 7 minute wash duration, wherein wash duration begins when the composition is contacted with water.
